(19) United States
(12) Patent Application Publication
(10) Pub. No.: US 2016/0287188 A1
(43) Pub. Date: Oct. 6, 2016

(54) INTRAVAGINAL SENSOR AND METHODS FOR SELECTING AN INSEMINATION TIME

(71) Applicant: Precise.ai inc., Kensington (CA)

(72) Inventors: Matthew B. CRANE, Kensington (CA); Melvin B. CRANE, Kensington (CA); Chellamma E. CRANE, Kensington (CA); Mary B. CRANE, Cornwall (CA)

(21) Appl. No.: 15/084,869

(22) Filed: Mar. 30, 2016

(30) Foreign Application Priority Data
Mar. 30, 2015 (CA) 2886522

(51) Int. Cl. A61B 5/00; A61B 5/01; A61B 5/11; A61B 5/053

(52) U.S. Cl. CPC A61B 5/7275; A61B 5/4337; A61B 5/6867; A61B 5/6879; A61B 5/0538; A61B 5/01; A61B 5/11; A61B 5/0022; A61B 5/7282; A61B 5/742

(57) ABSTRACT

A method and device for prediction of an insemination time most likely to result in conception in a mammal using a wireless intravaginal sensor is disclosed.

### BACKGROUND OF THE INVENTION

The present invention relates to improvements in and/or relating to the detection of animal estrus and intravaginal devices useful therein together with related means and methods.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

The present disclosure generally relates to the invention of intravaginal devices for sensing and transmitting physiological data from inside a mammalian vagina. The intravaginal devices can be inserted into a mammalian vagina over a period of time near or during which an estrus cycle could occur at least once. The intravaginal devices are able to retain themselves within the vaginal cavity.

One embodiment provides an intravaginal device having a motion sensor, impedance sensor, temperature sensor and a processor in a housing. The processor configured for processing sensor data. The sensor data collected by the device is processed and may be transmitted to a receiving system by a wireless transceiver. The receiving system calculates physiological and behavioural parameters related to the estrus cycle of that animal. Upon request the receiving system provides a qualified prediction for a time to apply artificial insemination. The receiving system additionally provides alerts of changes in the physiological status of the animal. In some embodiments some or all of the calculation of physiological and behavioural parameters that relate to the estrus cycle of that animal are performed within the intravaginal device instead of the receiving system.

Various refinements of the features noted above may exist in relation to the presently disclosed embodiments. Additional features may also be incorporated in these various embodiments as well. These refinements and additional features may exist individually or in any combination. For instance, various features discussed below in relation to one or more of the illustrated embodiments may be incorporated into any of the above-described embodiments alone or in any combination. Again, the brief summary presented above is intended only to familiarize the reader with certain aspects and contexts of embodiments of the present disclosure without limitation to the claimed subject matter.

### DESCRIPTION OF THE RELATED ART

Since the widespread adoption of the use of artificial insemination with frozen semen in domestic animals, the decision of when to apply insemination to the animal has become a matter requiring a great deal of attention from animal owners and farm operators. The two main benefits of artificial insemination with frozen semen are the ability to dilute the semen to enable insemination of many animals with the sperm from a single ejaculation and the portability of frozen semen that enables wide dispersion of the genetic material. However, both using lower sperm numbers and subjecting sperm cells to the stress of a freeze/thaw cycle narrow the effective insemination window. Recently the application of sex sorting technology to the semen of dairy bulls has resulted in an even more restricted effective breeding window.

In cattle the opportunity to inseminate normally occurs every 18 to 24 days and lasts about twelve hours. The most basic method of determining the time to inseminate cattle is to observe the onset of the behaviour of standing to be mounted by other cattle. Due to factors related to higher milk production the behavioural signs of estrus in dairy cattle have diminished and are sometimes totally absent. Modern housing systems and large herd sizes are factors that further reduce the effectiveness of insemination based on observed behaviour.

In the prior art, non-electronic devices which assist the operator with the timing of artificial insemination may be applied to the tail head of a cow. The discussed devices change color with the weight or abrasive forces applied during mounting behaviour. An example of this is the system disclosed by [U.S. Pat. No. 6,708,648 B2] and other similar methods such as chin ball markers, dye-release mount detectors, and tail chalking. Disadvantages of the discussed systems is the use is limited to animals that are in a loose housing enclosure so that the animals are free to show standing to be mounted behaviour, and visual observation of the changes in the device is required and an estimate of the best time to inseminate is made based on when that observation occurred.

Other known devices use motion sensors to assist with breeding. Cows are known to increase walking activity especially during the early stages of estrus. A number of known devices are in use that measure walking activity or head or neck or leg movements by use of motion sensors in the form of accelerometers and gyroscopes attached to the animal such as that disclosed by [U.S. Pat. No. 8,111,166 B2]. The discussed devices typically transmit activity data from a sensor to a receiver that records, analyses and displays the period of increased activity upon which a decision of when to inseminate is made by the farm operator. While these devices provide useful information on the onset of estrus in dairy animals their usefulness is limited to animals that are loosely housed and able to display outward signs of estrus. These devices are also prone to error in their inability to distinguish animals showing sympathetic behaviour or that are influenced by other animals participating in estrus activity. The onset and duration of increased activity is influenced by metabolic factors related to milk production, housing conditions and the animals health which makes prediction of timing of ovulation imprecise.

Body temperature taken over a period of time can be used to pinpoint the time of ovulation in mammals. [WO 2013162750 A1] discloses a device for identifying the time of ovulation in humans; however, a series of temperature readings must be made over a number of days at a specified time of day. In cattle rectal temperatures near the anal sphincter are not reliable and must be taken by deep rectal or vaginal probe. Devices are know that are applied by forced ingestion in dairy cattle that reside in the fore stomach and wirelessly transmit temperature readings to receiving devices outside of the animal's body, such as the device disclosed by [U.S. Pat. No. 8,640,712 B2]. These devices are limited because temperature in the fore stomach of ruminants is affected by the ingestion of large quantities of cold water and food as well as by the degree of microbial fermentation occurring at the time. These devices are further limited because algorithmic filtering of the data from these devices can yield useful temperature information relating to disease or infection but does not have enough sensitivity to detect temperature variation due to ovulation.

The electrical characteristics of the mammalian vagina are known to change near the time of ovulation. Handheld electrical impedance measurement devices are known, such as that disclosed by [U.S. Pat. No. 6,080,118 A]. In fur production with Arctic foxes (Vulpes lagopus) and Red foxes (Vulpes vulpes), a probe known as a rut gauge is inserted daily, deep in the vagina, to measure electrical impedance and is a gold standard for determining the best time to breed or to apply artificial insemination. Similar devices have been used in cattle but have not been accepted because the repeated application of the probe is not well tolerated by the animal and the labour required to probe eligible animals on a daily basis is more time consuming than other forms of estrus detection and is generally not compatible with modern farming practices.

Other relevant prior art includes [U.S. Pat. No. 5,109,865] and [U.S. Pat. No. 4,498,481]. The discussed systems, however, lack the ability to make automated measurements. For the discussed systems this means a significant amount of manual labour and record keeping is required to operate them. Other partially automated devices are known that rely on external transmitters affixed to a tail. This limits the application of the discussed devices to tailed animal. Tail mobility is critical in cases of environmental stress and for cow comfort, a problem not addressed in the prior art.

### BRIEF DESCRIPTION OF THE DRAWINGS

In the course of the detailed description to follow, reference will be made to the attached drawings. These drawings show different aspects of the present inventions and, where appropriate, reference numerals illustrating like structures, components, materials and/or elements in different figures are labeled similarly. It is understood that various combinations of the structures, components, and/or elements, other than those specifically shown, are contemplated and are within the scope of the present inventions.

Moreover, there are many inventions described and illustrated herein. The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, certain permutations and combinations are not discussed and/or illustrated separately herein.

FIG. 1 is a perspective view of an embodiment of the present invention, an intravaginal device which has a frame enclosing a space for accommodating an electronics package 207. FIG. 1 illustrates the intravaginal device as having a smooth, sleek and rounded exterior appearance 105,202,209 while at the same time including one or more sensors.

FIG. 2 is a perspective view of an embodiment of the present invention with a portion of the frame removed to reveal the enclosing space and electronics package 207.

FIG. 3 illustrates an example of the intravaginal sensing device in communication with a remote device.

Embodiments of the frame include a portion formed as a helix 102 or set of arms 203 as a means of retaining the device within the vagina. In some embodiments this is attached to a distal end of the frame enclosure.

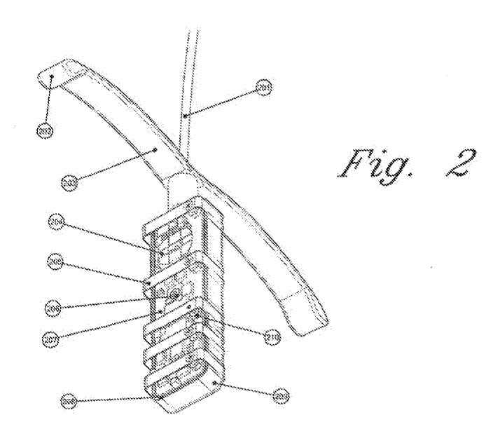

Referring now to FIG. 2 the interior of the intravaginal device will be discussed. The intravaginal device assembly includes the internal space of the frame which is made fluid tight. In some embodiments a seal of a nitrile-containing polymer or compressible material is placed between parts of the frame 208 to provide a means of hermetically sealing the internal space of the frame. In some embodiments the internal space includes, or has mounted therein, induction charging coil 204, rechargeable battery (not shown) in electrical connection with the induction coil 204.

### DETAILED DESCRIPTION

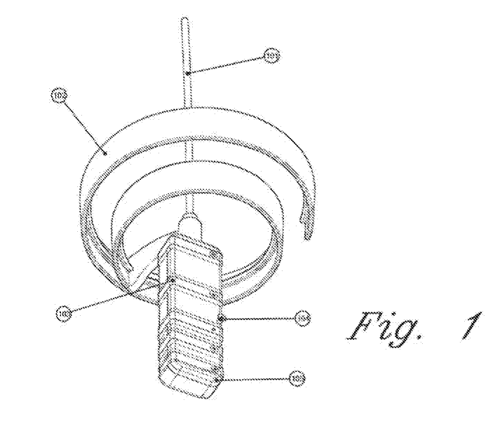

As shown in FIG. 1 the presently preferred embodiment of the invention includes the intravaginal device. The hermetically sealed space encloses an electronics package 207 for sensing, and processing data collected from the vaginal cavity.

Regarding the material and dimensions of the frame, numerous options are available, as will be appreciated by one skilled in the art. In some embodiments, the material of the frame is comprised of a poly(tetrafluroethylene), in other embodiments it is comprised of a poly(ethylene). In many embodiments a diversity of polymers are used. In all embodiments the frame material in contact with the vaginal cavity is bio-compatible, such that it has the quality of not having toxic or injurious effects on biological systems. In some embodiments elements of the frame in contact with the vaginal wall are comprised with mucoadhesive particles in order to improve vaginal retention.

In an embodiment, an element of the frame comprises the means to retain the device within the vagina that consists of a portion formed as a helix 102 or set of arms 203. In some embodiments this is attached to a distal end of the frame enclosure. One skilled in the art will appreciate that the size of the helical coil and arms and the number of arms may vary to accommodate particular vaginal cavity shapes.

In one embodiment a nub feature on a distal end of the frame enclosure is attached interchangeably to variously sized elements of the frame. In these embodiments frame elements comprising the means to retain the device in the vagina can be chosen to match the vaginal cavity size of a target species, without reliance on a tail for support or anchor.

In exercise of some embodiments of the intravaginal device a tubular shaped applicator is used to place the device within the vaginal cavity. In embodiments of the device used with an applicator the applicator has an open end formed to hold an intravaginal device and a plunger disposed within the tube. When an embodiment of the device is placed in an appropriate applicator tube the retaining arms or retaining coil are compressed. For such an applicator containing an intravaginal device the insertion into the vaginal cavity and depression of the plunger releases the intravaginal device into the vaginal cavity so that the retaining arms or retaining coil are left exerted against the vaginal cavity and the pull cord is left visible outside the vagina, upon removal of the applicator.

In the presently preferred embodiments elements of the frame enclose a hermetically sealed space housing an electronics package which includes printed circuit board assemblies. In one embodiment the printed circuit board assemblies logic components includes a microprocessor, wireless transceiver, thermometer, impedance measurement, electrode selection circuit, motion sensor and non-volatile memory storage. The motion sensor can be one of or include an accelerometer, or a magnetometer, or a gyroscope, or a linear motion sensor, or an angular motion sensor, or a multi-axis motion sensor, or a combination of two or more thereof.

In one embodiment, microprocessor functions in conjunction with the various logic components 306. The microprocessor can, in one embodiment, provide the functionality of any one or all of the logic components. In other embodiments, multiple chips can be used to separate the processing performed by any one of the logic components and the processor. Sensors can communicate via a bus with the processor and/or the logic components. The storage 305 is also in communication with the bus for providing storage of the sensor data processed or logged by the intravaginal device. Battery is provided for providing power to the intravaginal device.

The intravaginal device FIG. 1 can communicate with a remote device using the wireless transceiver. The wireless transceiver will allow the intravaginal device to communicate using a wireless connection, which is enabled by wireless communication logic. The wireless communication logic can be in the form of a circuit having radio communication capabilities. The radio communication capabilities can be in the form of a Wi-Fi connection, a low-energy Bluetooth connection, a Bluetooth connection, or any other form of wireless tethering or near field communication. In some embodiments, an antenna for the wireless transceiver is contained in a cable extending out of a distal end of the device so that a radiating element is exposed outside the vagina. In some embodiments the antenna cable acts as a pull cord that allows the device to be removed from the vaginal cavity 201, 101.

Other methods disclosed herein could be carried out in part or in full by a server or remote device. In example embodiment, a server or remote device may receive intravaginal sensor output data from an intravaginal device FIG. 1, operating in an mammal, such as a plurality of impedance values captured using an impedance sensor. The sensor output data could be used by the server or remote device to determine one or more predicted physiological status and may additionally or alternatively determine one or more confidence levels that could be related to one or more likelihoods that the mammal match a predicted state. Other interactions between an intravaginal sensor device and a server are possible within the context of the disclosure.

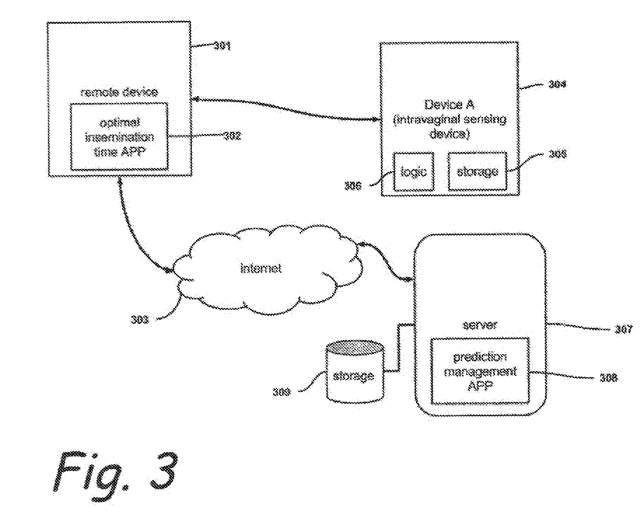

FIG. 3 illustrates an example of an intravaginal sensing device 304 in communication with a remote device 301. Remote device 301 is a computing device that is capable of communicating wirelessly with an intravaginal device 304 and with the Internet 303. Remote device 301 can support installation and execution of applications. Such applications can include an optimum insemination time application 302. Optimum insemination time application 302 can be downloaded from a server. The server 220 can be a specialized server or a server that provides applications to devices, such as an application store. Once the optimum insemination time application 302 is installed in the remote device 301, the remote device 301 can communicate or be set to communicate with intravaginal sensing device 304 (Device A). The remote device 301 can be a smartphone, a handheld computer, a tablet computer, a laptop computer, dongle computer, a desktop computer, or any other computing device capable of wirelessly interfacing with Device A 304 and the Internet 303. The remote device or server may have storage 309 used to access or create datasets of intravaginal sensor output data.

In one embodiment the electrodes are thin strips of conductive and non-reactive material such as stainless steel and are connected to the printed circuit board assembly 207 via a low-impedance connection 210, 206. In another embodiment the electrodes are constructed out of a polymer with a conductive coating applied. In some embodiments the electrode surface is coated to facilitate bio-compatibility. Embodiments of the invention with a impedance sensor will have a number of electrodes 205, 103 with a low impedance connection 206, 210 to the internal space of the frame. One skilled in the art will appreciate the many possible arrangements of electrodes. In some embodiments the discussed connection may also serve to mechanically secure printed circuit board assemblies 207 to the frame.

During active impedance measurement an embodiment of the invention comprises at least one transmitting electrode and one or more receiving electrodes. Embodiments with impedance sensing logic comprise switching logic to select a low-impedance path from the impedance sensing logic to a particular electrode. The innovation includes a means of using the microprocessor to control the switch coupled to receiving and transmitting electrodes so that the impedance between various electrodes can be calculated.

The impedance sensing logic can be connected to a number of electrodes through the switching logic. The impedance sensing logic can be configured to measure the impedance with the help of at least two such electrodes. The impedance indicates the impedance across tissues or fluid within a portion of the vaginal cavity. How to measure such an impedance is known to those skilled in the art, for example from some of the above-mentioned documents. For example, the control circuit can be configured to inject an alternating current between the electrodes and to measure a voltage between the electrodes. However, other combinations of electrode surfaces can be used for the impedance measurement. However, the impedance sensing logic is preferably set up such that the variation of the measured impedance is related to the variations of vaginal mucosa present in the vaginal cavity.

In order to determine the occasions to carry out impedance sensing an embodiment of the device may rely on motion sensor data to detect an increase in activity. In one embodiment the processing logic computes a change in motion sensor output data indicative of the onset of estrus and a corresponding confidence level. In some embodiments, in order to determine the occasions to carry out impedance sensing, an embodiment of the device may rely on a confidence level derived from another sensor. In one embodiment the selection of the transmitting and receiving electrode may depend on a confidence level derived from the same or different transmitting and receiving electrodes.

What is claimed:

1. An intravaginal sensing device capable of being applied into the vaginal cavity of a mammal, retainable therein, and then to be withdrawable therefrom,
said device having a frame comprised of a plurality of walls that surround a hermetically sealed internal space, such that the frame includes a means of retaining itself in the vagina comprising one of
two or more arms capable of being moved towards each other against the resilience thereof so as, in its vaginal cavity retainable form, to provide an outward pressure reliant upon the vaginal cavity contained splayed condition of the arms thereof,
a helical coil that upon compression exerts outward pressure radially, in its vaginal cavity retainable form, against the resilience of the vaginal cavity wall;
wherein its vaginal cavity retainable form said frame exposes a plurality of electrodes to the environment of the vaginal cavity;
where said electrodes are electrically coupled to the internal space of the frame with a low-impedance interconnection
and wherein said device
upon vaginal insertion into such a mammal for which it is sized and targeted, detects and reports sensor output data relating to the status of said mammal.

2. The device of claim 1, where said frame is comprised of a plurality of bio-compatible and electrically insulating polymers.

3. The device of claim 1, where disposed within the internal space of the frame is
a. radio transceiver circuitry,
b. processing circuitry that is
i. communicatively coupled to a plurality of sensors from the group of impedance, temperature, motion,
ii. communicatively coupled to a memory storage,
iii. communicatively coupled to radio transceiver circuitry;
c. a means to sense electrical impedance at a plurality of frequencies,
d. a means for selecting a plurality of electrodes for the purposes of impedance sensing,
e. a means of sensing one or both of temperature and motion.

2. The device of claim 1 where the means to withdraw the device from the vagina is comprised of a radio frequency antenna.

3. The device of claim 1 where the means to receive current for purposes of energy storage comprises a single coil for inductively receiving current

4. A method of displaying a breeding time of a mammal, for purposes of insemination, comprising:
by a processor
a. collecting sensor output data from an intravaginal sensing device, where said sensor output data is from the group of temperature, motion, and impedance;
b. where said sensor output data is collected over a period of time of less then 30 days from within a mammals intravaginal cavity;
c. correlating variations within said sensor output data with the onset of estrus in said mammal, at a plurality of times, so as to create, for each available sensor in the group of temperature, motion, and impedance, a dataset of probabilities that the onset of estrus has occurred;
d. for each of said datasets, computing a confidence level;
e. modifying a series of mathematical operations such that said modifications are dependant on said confidence levels;
f. computing said mathematical operations to generate one prediction and a corresponding confidence level for a time to breed likely to result in conception.

5. The method of claim 4 wherein said processor is contained within an intravaginal sensing device.

6. The method of claim 4, wherein displaying a breeding time of a mammal comprises displaying a corresponding confidence level.

7. The method of claim 4, wherein displaying a breeding time comprises displaying a breeding time only if the confidence level is at or above a threshold.

8. A method of selecting a breeding time of a mammal, for purposes of insemination, utilizing external sensors associated with the vaginal cavity of said mammal, comprising:
by a processor
a. collecting sensor output data from at least one sensor associated with the vaginal cavity of said mammal and in a frame, said frame residing within the vaginal cavity of said mammal, said sensor from the group of temperature, motion, and impedance, where said sensor output data is indicative of the onset of estrus within said mammal;
b. for said sensor output data, creating a dataset of probabilities that the onset of estrus has occurred for said mammal at a plurality of times;
c. selecting a series of mathematical operations for derivation of a predicted time for onset of estrus of said mammal from said dataset
d. computing said mathematical operations to create first a predicted time for onset of estrus and second a breeding time.

9. The method of claim 9, wherein said processor is contained within an intravaginal sensing device.
